(19) United States
(12) Patent Application Publication
(10) Pub. No.: US 2006/0083706 A1
(43) Pub. Date: Apr. 20, 2006

(54) Haircare composition comprising at least one electrophilic monomer and micro- or nanoparticles of at least one solid lubricant

(76) Inventors: Gabin Vic, Venette (FR); Aude Livoreil, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/248,331

(22) Filed: Oct. 13, 2005

Related U.S. Application Data
(60) Provisional application No. 60/645,594, filed on Jan. 24, 2005.

(30) Foreign Application Priority Data
Oct. 13, 2004 (FR) 0411047

(51) Int. Cl. A61K 8/81

(52) U.S. Cl. 424/70.16

(57) ABSTRACT

Disclosed herein is a haircare composition comprising, in a cosmetically acceptable medium, at least one electrophilic monomer and micro- or nanoparticles of at least one solid lubricant.

This application claims benefit of U.S. Provisional Application No. 60/645,594, filed Jan. 24, 2005, the contents of which are incorporated herein by reference. This application also claims benefit of priority under 35 U.S.C. § 119 to French Patent Application No. 04 11047, filed Oct. 13, 2004, the contents of which are also incorporated by reference.

Disclosed herein are novel compositions for giving hair volume, comprising in-situ polymerizable monomers, a cosmetically acceptable medium, and micro- or nanoparticles of at least one solid lubricant. Also disclosed are possible uses of these compositions for the haircare treatment of keratin materials.

As used herein, the term “keratin materials” means the hair.

Numerous styling products exist for giving the hair volume. One drawback associated with these products, which are usually based on film-forming polymers, lies in the fact that the cosmetic effect may disappear after the first shampoo wash.

Permanent-waving treatments of the hair are also known. These treatments may use a reducing agent and an oxidizing agent, and may require the placing of the hair under mechanical tension using rolling equipment, in order to impart a shape.

These processes, which effectively may make it possible to increase the volume of the head of hair, may have the drawback of modifying the level of curliness of the hair and of degrading the feel of the hair, without giving sufficient sheen and smoothness.

It thus appears necessary to develop compositions for increasing the volume of the head of hair without modifying the shape or feel of the hair, while at the same time being shampoo-fast, and giving the hair sheen and smoothness.

Disclosed herein are novel compositions which may in some embodiments overcome at least one these drawbacks.

It has been discovered that by using electrophilic monomers as described in French Patent Application FR 2 840 208 and micro- or nanoparticles of at least one solid lubricant, it may be possible to give the hair style, volume, and add sheen and smoothness to the hair, without degrading the hair and without the hairs adhering together. In addition, these cosmetic properties may withstand several shampoo washes.

In addition to the volume, certain particles also may make it possible to give the head of hair long-lasting sheen, body, mass, and optical effects.

It has also been discovered that by applying a composition comprising such monomers and micro- or nanoparticles of at least one solid lubricant to the hair, a long-lasting coat covering the hair may be formed in situ.

One embodiment disclosed herein is thus a haircare composition comprising, in a cosmetically acceptable medium, at least one electrophilic monomer and micro- or nanoparticles of at least one solid lubricant.

Another embodiment disclosed herein is the use, for the treatment of the hair, of a composition comprising, in a cosmetically acceptable medium, at least one electrophilic monomer and micro- or nanoparticles of at least one solid lubricant.

Also disclosed herein is a kit comprising a first composition comprising at least one electrophilic monomer (present in an amount that may range from 0.5% to 50% by weight relative to the total weight of the first composition) and at least one polymerization inhibitor chosen from anionic and free-radical polymerization inhibitors (present in an amount that may range from 10 ppm to 5% by weight relative to the total weight of the first composition), and also a second composition comprising, in a cosmetically acceptable medium, micro- or nanoparticles of at least one solid lubricant (present in an amount that may range from 0.001% to 4% by weight relative to the total weight of the second composition).

Other embodiments will become apparent on reading the description and the examples that follow.

As used herein, the term “nanoparticle” means any particle whose elementary size ranges from 1 nm to 999 nm, and the term “microparticle” means any particle whose elementary size ranges from 1 μm to 300 μm.

This nano- or microparticle may be in a form chosen from spheres, needles, flakes, platelets, tubes, fibers, cubes, prisms, and irregular forms.

As used herein, the term “particle size” means the distance between the two most distant points of the particle.

As used herein, the term “solid lubricant” means any solid material that reduces the friction and/or the wear of two surfaces in contact, moving relative to each other. Such compounds are described, for example, in Ullmann's Encyclopaedia of Industrial Chemistry 2002, in the chapter “Solid Lubricant” written by Christian Busch; and in Kirk Othmer's Encyclopaedia of Chemical Technology, in the article “Lubrication and Lubricants”, chapter 3, “Solid Film Lubricants”, written by E. R. Boares.

Nano- or microparticles that may be mentioned, in a non-limiting manner, include electroluminescent semiconductive nanoparticles (or quantum dots), nano- or microfibrils, microplatelets, latices, nanotubes, adhesive microobjects, and expandable particles.

These particles may be mineral or organic.

In certain embodiments, the nano- or microparticles are chosen from particles of:

graphite, graphite fluoride, KC₈ (potassium graphite), LiC₈ (lithium graphite)

molybdenum disulfide (MOS₂);

boron nitride;

tin disulfide (SnS₂);

BaSi₂;

silicate Si₂O₅;

titanium disulfide (TiS₂);

Li₂Si₂O₅;

cerium fluoride (CeF₃);

kaolinite;

talc;

pyrophyllite (Al₂Si₄O₁₀(OH)₂);

mica;

zirconium chloride (ZrCl₂);

muscovite;

silver sulfate (Ag₂SO₄);

montmorillonite;

silicone beads;

borax (Na₂B₄O₇);

polyterephthalates;

perfluoroalkoxy (PFA) polymers;

fluoroethylene propene (FEP) copolymer; and

nylon

The micro- or nanoparticles of at least one solid lubricant may be present in the composition in an amount ranging from 0.0001% to 40% by weight, such as from 0.001% to 20% or from 0.01% to 10%, by weight relative to the total weight of the composition.

In certain embodiments, the micro- or nanoparticles of at least one solid lubricant are minerals.

As used herein, the term “electrophilic monomer” means a monomer capable of polymerizing by anionic polymerization in the presence of at least one nucleophilic agent, for instance the hydroxide ions (OH⁻) contained in water.

As used herein, the term “anionic polymerization” means the mechanism defined in the book “Advanced Organic Chemistry”, third edition, by Jerry March, pages 151 to 161.

At Least One Electrophilic Monomer

As disclosed herein, the at least one electrophilic monomer may be chosen from monomers capable of polymerizing on the keratin fibers under cosmetically acceptable conditions. For example, the polymerization of the at least one electrophilic monomer may be performed at a temperature of less than or equal to 80° C., such as a temperature ranging from 10 to 80° C. or from 20 to 80° C., wherein the temperature does not prevent the application from being completed by drying under a drying hood, blow-drying, or treating with a flat iron or a crimping iron.

The at least one electrophilic monomer present in the compositions disclosed herein may be chosen from:
the benzylidene malononitrile derivatives (A), 2-(4-chloro-benzylidene)malononitrile (A1), ethyl 2-cyano-3-phenylacrylate (B), and ethyl 2-cyano-3-(4-chlorophenyl)acrylate (B1) described, for example, in Sayyah, J. Polymer Research, 2000, p. 97: 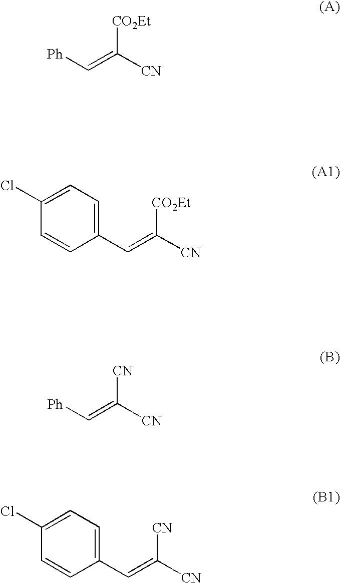
methylidenemalonate derivatives, for instance:
diethyl 2-methylenemalonate (C) described, for example, in Hopff, Makromoleculare Chemie, 1961, p. 95, De Keyser, J. Pharm. Sci, 1991, p. 67 and Klemarczyk, Polymer, 1998, p. 173: 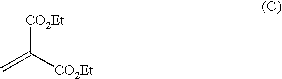
   ethyl 2-ethoxycarbonylmethylenecarbonylacrylate (D) described, for example, in Breton, Biomaterials, 1998, p. 271 and Couvreur, Pharmaceutical Research, 1994, p. 1270: 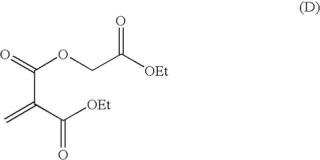
   itaconate and itaconimide derivatives, for instance:
dimethyl itaconate (E), described, for example, in Bachrach, European Polymer Journal, 1976, p. 563: 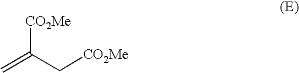
   N-butyl itaconimide (F), N-(4-tolyl) itaconimide (G), N-(2-ethylphenyl) itaconimide (H), N-(2,6-diethylphenyl) itaconimide (I) described, for example, in Wanatabe, J. Polymer Science: Part A: Polymer chemistry, 1994, p. 2073:

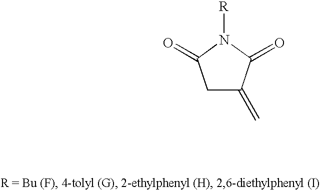

the derivatives methyl α-(methylsulfonyl)acrylate (K), ethyl α-(methylsulfonyl)acrylate (L), methyl α-(tert-butylsulfonyl)acrylate (M), tert-butyl α-(methylsulfonyl)acrylate (N), and tert-butyl α-(tert-butylsulfonyl)acrylate (O), described, for example, in Gipstein, J. Org. Chem, 1980, p. 1486. and the derivatives 1,1-bis(methylsulfonyl)ethylene (P), 1-acetyl-1-methylsulfonylethylene (O), methyl α-(methylsulfonyl)vinylsulfonate (R), and α-methylsulfonylacrylonitrile (S), described, for example, in U.S. Pat. No. 2,748,050 to Shearer, et al.:

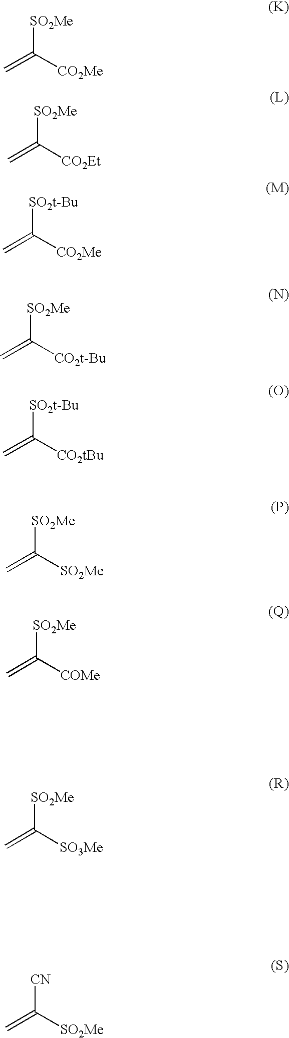

the methyl vinyl sulfone (T) and phenyl vinyl sulfone (U) derivatives described, for example, by Boor, J. Polymer Science, 1971, p. 249:

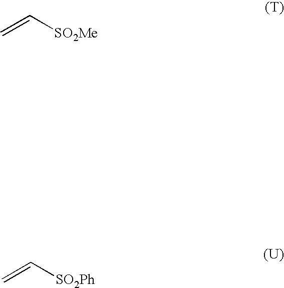

the phenyl vinyl sulfoxide derivative (V) described, for example, in Kanga, Polymer preprints (ACS, Divison of Polymer Chemistry), 1987, p. 322:

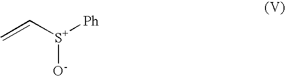

the derivative 3-methyl-N-(phenylsulfonyl)-1-aza-1,3-butadiene (W) described, for example, in Bonner, Polymer Bulletin, 1992, p. 517:

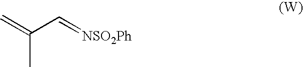

acrylate and acrylamide derivatives, for instance:
N-propyl-N-(3-triisopropoxysilylpropyl)acrylamide (X) and N-propyl-N-(3-triethoxysilylpropyl)acrylamide (Y) described, for example, in Kobayashi, Journal of Polymer Science, Part A: Polymer Chemistry, 2005, p. 2754: 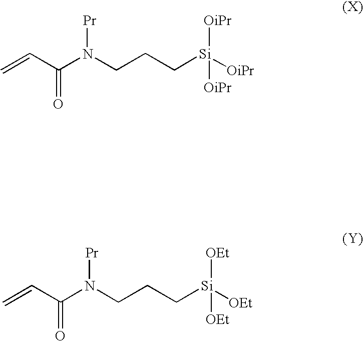
   2-hydroxyethyl acrylate (Z) and 2-hydroxyethyl methacrylate (AA) described, for example, in Rozenberg, International Journal of Plastics Technology, 2003, p. 17: 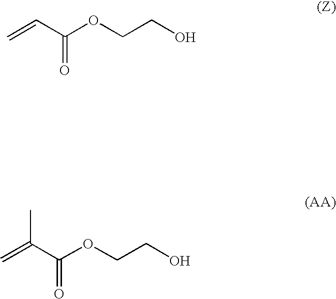
   N-butyl acrylate (AB) described, for example, in Schmitt, Macromolecules, 2001, p. 2115, and tert-butyl acrylate (AC) described, for example, in Ishizone, Macromolecules, 1999, p. 955:

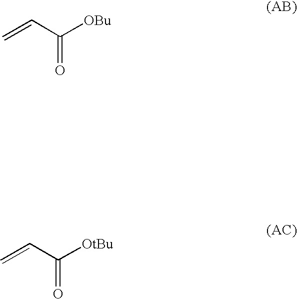

The at least one electrophilic (or electron-withdrawing) monomer that may be used according to certain embodiments disclosed herein may be cyclic or linear. When it is cyclic, the electron-withdrawing group may be exocyclic, i.e., it does not form an integral part of the cyclic structure of the monomer.

According to one embodiment, the at least one electrophilic monomer comprises at least two electron-withdrawing groups.

As examples of monomers comprising at least two electron-withdrawing groups, mention may be made of the monomers of formula (A):

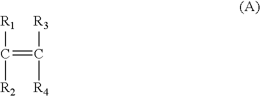

in which:

R₁ and R₂ are, independently of each other, chosen from sparingly or non-electron-withdrawing groups (sparingly or non-inductive-withdrawing) such as:

hydrogen,

saturated or unsaturated, linear, branched, or cyclic hydrocarbon-based groups, for example groups containing from 1 to 20 or from 1 to 10 carbon atoms, and optionally comprising at least one atom chosen from nitrogen, oxygen, and sulfur atoms, and optionally substituted with at least one group chosen from —OR, —COOR, —COR, —SH, —SR, —OH, and halogen atoms,

modified or unmodified polyorganosiloxane residues, and

polyoxyalkylene groups;

R₃ and R₄ are, independently of each other, chosen from electron-withdrawing (or inductive-withdrawing) groups, which may be chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH groups, linear or branched alkenyl groups, linear or branched alkynyl groups, C₁-C₄ monofluoroalkyl groups, polyfluoroalkyl groups, aryl groups such as phenyl, and aryloxy groups such as phenoxyloxy; and

R is chosen from saturated or unsaturated, linear, branched, or cyclic hydrocarbon-based groups which may contain from 1 to 20 or from 1 to 10 carbon atoms, and optionally comprise at least one atom chosen from nitrogen, oxygen, and sulfur atoms, and optionally substituted with at least one group chosen from —OR′, —COOR′, —COR′, —SH, —SR′, —OH, halogen atoms, and polymer residues, wherein R′ is chosen from C₁-C₁₀ alkyl radicals, this polymer optionally being obtained by free-radical polymerization, by polycondensation, or by ring opening.

As used herein, the term “electron-withdrawing or inductive-withdrawing group” means any group that is more electronegative than carbon. Reference may be made to the publication P. R. Wells, Prog. Phys. Org. Chem., Vol 6, 111 (1968).

As used herein, the term “sparingly or non-electron-withdrawing group” means any group whose electronegativity is less than or equal to that of carbon.

The alkenyl or alkynyl groups may contain from 2 to 20 carbon atoms, for example from 2 to 10 carbon atoms.

As saturated or unsaturated, linear, branched, or cyclic hydrocarbon-based groups that may containing from 1 to 20 carbon atoms, for example 1 to 10 carbon atoms, mention may be made of linear or branched alkyl, alkenyl, and alkynyl groups, such as methyl, ethyl, n-butyl, tert-butyl, isobutyl, pentyl, hexyl, octyl, butenyl, and butynyl groups; cycloalkyl groups; and aromatic groups.

Examples of substituted hydrocarbon-based groups that may be mentioned include hydroxyalkyl and polyhaloalkyl groups.

Examples of unmodified polyorganosiloxanes that may be mentioned include polyalkylsiloxanes such as polydimethylsiloxanes, polyarylsiloxanes such as polyphenylsiloxanes, and polyarylalkylsiloxanes such as polymethylphenylsiloxanes.

Among the modified polyorganosiloxanes that may be mentioned are polydimethylsiloxanes containing at least one group chosen from polyoxyalkylene, siloxy, silanol, amine, imine, and fluoroalkyl groups.

Among the polyoxyalkylene groups that may be mentioned are polyoxyethylene groups and polyoxypropylene groups, which may containing 1 to 200 oxyalkylene units.

Among the monofluoroalkyl groups and polyfluoroalkyl groups that may be mentioned are groups such as —(CH₂)ₙ—(CF₂)ₘ—CF₃ and —(CH₂)ₙ—(CF₂)ₘ—CHF₂, wherein n ranges from 1 to 20 and m ranges from 1 to 20.

The substituents R₁ to R₄ may optionally be substituted with a group having cosmetic activity. The cosmetic activities that may be mentioned include coloring, antioxidant, UV-screening, and conditioning functions.

As examples of groups having a coloring function, mention may be made of azo, quinone, methine, cyanomethine, and triarylmethane groups.

As examples of groups having an antioxidant function, mention may be made of butylhydroxyanisole (BHA), butylhydroxytoluene (BHT), and vitamin E groups.

As examples of groups having a UV-screening function, mention may be made benzophenone, cinnamate, benzoate, benzylidenecamphor, and dibenzoylmethane groups.

As examples of groups having a conditioning function, mention may be made of cationic groups and fatty ester groups.

Among the at least one electrophilic monomer mentioned above, mention may be made of the monomers of the cyanoacrylate family and the derivatives thereof of formula (B):

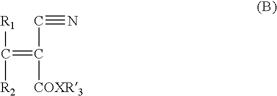

wherein X is chosen from NH, S, and O;

R₁ and R₂ having the same meanings as above; and

R′₃ is chosen from hydrogen and radicals R as defined for formula (A).

In certain embodiments, X is O.

Compounds of formula (B) that may be mentioned include the monomers:

a) belonging to the family of C₁-C₂₀ polyfluoroalkyl 2-cyanoacrylates such as:

the ester 2,2,3,3-tetrafluoropropyl 2-cyano-2-propenoate of formula:

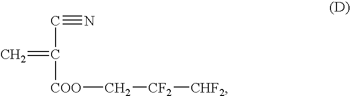

and the ester 2,2,2-trifluoroethyl 2-cyano-2-propenoate of formula:

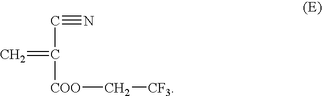

b) the C₁-C₁₀ alkyl and (C₁-C₄ alkoxy)(C₁-C₁₀ alkyl) cyanoacrylates.

Mention may also be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

According to certain embodiments, the monomers b) may be used.

The monomers of formula F and mixtures thereof may also be mentioned:

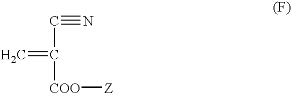

wherein Z is chosen from —(CH₂)₇—CH₃; —CH(CH₃)—(CH₂)₅—CH₃; —CH₂—CH(C₂H₅)—(CH₂)₃—CH₃; —(CH₂)₅—CH(CH₃)—CH₃; and —(CH₂)₄—CH(CH₂H₅)—CH₃.

The at least one electrophilic monomer used in accordance with certain embodiments disclosed herein may be covalently bonded to supports such as polymers, oligomers, and dendrimers. The polymer or the oligomer may be linear, branched, in comb form, or in block form. The distribution of the at least one electrophilic monomer over the polymeric, oligomeric, or dendritic structure may be random, in an end position, or in the form of blocks.

As used herein, the term “cosmetically acceptable medium” means a medium that is compatible with the hair.

The cosmetically acceptable medium may be anhydrous. As used herein, the term “anhydrous medium” means a medium containing less than 1% by weight of water relative to the total weight of the composition.

The cosmetically acceptable medium may be chosen from mediums chosen from at least one of organic oils; silicones such as volatile silicones, amino silicone gums and oils, non-amino silicone gums and oils; mineral oils; plant oils such as olive oil, castor oil, rapeseed oil, coconut oil, wheatgerm oil, sweet almond oil, avocado oil, macadamia oil, apricot oil, safflower oil, candlenut oil, camelina oil, tamanu oil, and lemon oil; waxes; and organic compounds such as C₅-C₁₀ alkanes, acetone, methyl ethyl ketone, esters of C₁-C₂₀ acids and esters of C₁-C₈ alcohols such as methyl acetate, butyl acetate, ethyl acetate, and isopropylmyristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide, and C₁₀-C₃₀ fatty alkyl esters such as C₁₀-C₃₀ fatty alkyl benzoates.

In certain embodiments, the organic compounds are chosen from compounds that are liquid at a temperature of 25° C. and at 105 Pa (760 mmHg).

The compositions used in accordance with certain embodiments disclosed herein may have an amount of electrophilic monomer ranging from 0.001% to 80% by weight, such as from 0.1% to 40% by weight or from 1% to 20% by weight, relative to the total weight of the composition.

At least one polymerization inhibitor, such as anionic and free-radical polymerization inhibitors, may also be introduced into the compositions disclosed herein, in order to enhance the stability of the composition over time. In a non-limiting manner, the following polymerization inhibitors may be mentioned: sulfur dioxide; nitric oxide; lactone; boron trifluoride; hydroquinone and derivatives thereof such as hydroquinone monoethyl ether and tert-butylhydroquinone (TBHQ); benzoquinone and derivatives thereof such as duroquinone; catechol and derivatives thereof such as t-butylcatechol and methoxycatechol; anisole and derivatives thereof such as methoxyanisole, hydroxyanisole, and butylhydroxyanisole; pyrogallol; 2,4-dinitrophenol; 2,4,6-trihydroxybenzene; p-methoxyphenol; hydroxybutyltoluene; alkyl sulfates; alkyl sulfites; alkyl sulfones; alkyl sulfoxides; alkyl sulfides, mercaptans; and 3-sulfonene. The alkyl groups may be chosen from groups containing 1 to 6 carbon atoms.

It is also possible to use mineral or organic acids, the latter comprising at least one group chosen from carboxylic and sulfonic groups, with a pKa ranging from 0 to 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di-, and trichloroacetic acid, salicylic acid, and trifluoroacetic acid.

The amount of the at least one polymerization inhibitor may range from 10 ppm to 20%, such as from 10 ppm to 5% or from 10 ppm to 1%, by weight relative to the total weight of the composition.

The hair treatment process disclosed herein comprises applying the composition described above to keratin materials, for example in the presence of at least one nucleophilic agent, with or without heating.

In certain embodiments, the at least one nucleophilic agent is water. This water may be provided by wetting beforehand.

It is also possible, in order to modify the reaction kinetics, to wet the keratin materials beforehand using an aqueous solution wherein the pH has been adjusted using a base, an acid, or an acid/base mixture. The acid and/or the base may be mineral or organic.

These two operations may also be performed after applying the composition.

It is also possible to modify the anionic polymerization kinetics by preimpregnating the keratin materials with at least one nucleophilic agent. The at least one nucleophilic agent may be used pure, as a solution, in the form of an emulsion, or may be encapsulated.

The nucleophilic agents capable of initiating the anionic polymerization are systems that may be known per se, which are capable of generating a carbanion on contact with a nucleophilic agent, such as the hydroxide ions contained in water. As used herein, the term “carbanion” means the chemical species defined in “Advanced Organic Chemistry”, third edition, by Jerry March, page 141.

The at least one nucleophilic agent may comprise at least one entity chosen from molecular compounds, oligomers, dendrimers, and polymers containing at least one nucleophilic function. In a non-limiting manner, nucleophilic functions that may be mentioned include the following functions: R₂N⁻, NH₂⁻, Ph₃C⁻, R₃C⁻, PhNH⁻, pyridine, ArS⁻, R—C≡C⁻, RS⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, and H₂O, wherein Ph represents a phenyl group; Ar represents an aryl group; and R is chosen from C₁-C₁₀ alkyl groups.

It is also possible to modify the anionic polymerization kinetics by preimpregnating the hair with at least one nucleophilic agent other than water.

To modify the anionic polymerization kinetics, it is also possible to increase the nucelophilicity of the hair via chemical conversion of the keratin material.

Examples that may be mentioned include reducing the disulfide bridges of which keratin is partly composed into thiols, before applying the composition disclosed herein. In a non-exhaustive manner, as reducing agents for the disulfide bridges of which keratin is partly composed, mention may be made of the following compounds:
anhydrous sodium thiosulfate,
powdered sodium metabisulfite,
thiourea,
ammonium sulfite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulfoxylate,
isooctyl thioglycolate,
dl-cysteine, and
monoethanolamine thioglycolate.

To modify the anionic polymerization kinetics, for example to reduce the rate of polymerization of the at least one electrophilic monomer disclosed herein, it may be possible to increase the viscosity of the composition. To do this, at least one polymer that has no reactivity towards the at least one electrophilic monomer may be added to the compositions disclosed herien. In this context, mention may be made, in a non-exhaustive manner, of poly(methyl methacrylate) (PMMA) and cyanoacrylate-based copolymers as described, for example, in U.S. Pat. No. 6,224,622.

In order to improve, inter alia, the adhesion of the poly(cyanoacrylate) formed in situ, the hair may be pretreated with polymers of any type, or a haircare treatment may be performed before applying the composition disclosed herein, for instance a haircare treatment chosen from direct dyeing, oxidation dyeing, permanent-waving, hair relaxing operations.

The application of the compositions contained in an aerosol device as disclosed herein may optionally be followed by rinsing. These compositions may be in various forms, such as in the form of lotions, sprays, and mousses, and may be applied in the form of a shampoo or a hair conditioner.

The mode of application may be in a single step or alternatively may be divided into successive steps. If the process includes several steps of application of active compositions, the steps may be as follows, in either order:

1. Applying to the hair the micro- or nanoparticles of at least one solid lubricant, present in an aqueous solution in an amount ranging from 0.05% to 40%, such as from 0.1% to 35% or from 0.25% to 25%, by weight relative to the total weight of the solution;

2. Applying to the wet hair the at least one electrophilic monomer, which is present in solution in an amount ranging from 0.05% to 30% by weight, such as from 0.01% to 50% by weight or from 0.1% to 20% by weight, relative to the total weight of the solution.

In addition to the active agent, the compositions disclosed herein may comprise at least one conventional haircare additive. The order of the first two steps may be inverted. The first step may be preceded by applying a haircare product, such as a care product, shampoo, conditioner, dyeing, bleaching, permanent-waving, or hair-relaxing product. Similarly, the last step may be succeeded by applying a haircare product. Each step may be interrupted by rinsing and/or drying. The drying may be performed under a drying hood, with a hairdryer and/or with a straightening iron.

Also disclosed herein is a process, including at least the two following steps:

a first step comprising applying a composition containing micro- or nanoparticles of at least one solid lubricant; and

a second step comprising applying a composition comprising at least one electrophilic monomer.

In certain embodiments, the first and the second steps are inverted.

Another embodiments disclosed herein is the use of the compositions described above for the haircare treatment of keratin materials, such as the hair. The compositions may be used for reinforcing the hair.

The examples that follow are intended to illustrate certain embodiments without, however, being limiting in nature. Tests were performed using the following compound: n-octyl 2-cyanoacrylate monomer, sold under the name Rite Lok® CON895 by the company Chemence.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

### EXAMPLES

Composition 1 (Comparative)

| | |
|---|---|
| 2-Octyl cyanoacrylate | 10% a.m. |
| Cyclopentasiloxane | 45% |
| Cyclopentasiloxane dimethicone | 45% |
| copolyol mixture | |

Composition 2

| | |
|---|---|
| 2-Octyl cyanoacrylate | 10% a.m. |
| Cyclopentasiloxane | 43% |
| Cyclopentasiloxane dimethicone | 43% |
| copolyol | |
| Boron nitride | 4% |

Mode of Application

Locks comprising 2.7 g of sensitized hair were wetted with 1 ml of water per lock. 2 g of the compositions described above were applied to these wet locks. After application, the locks of hair were dried under a drying hood for 30 minutes at 40° C.

For each lock, the feel and the sheen of the hair were evaluated by a panel of 10 people. A virgin lock of the same sensitization was used as reference. The tactile and visual evaluation of the various locks of hair was repeated with the same procedure after washing them 5 times successively with a shampoo sold under the name DOP camomile.

| | Nature of the | | |
|---|---|---|---|
| | treatment | Composition 1 | Composition 2 |
| Sensory | After application | Softness 3 | Softness 5 |
| evaluation | | Disentangling 3 | Disentangling 5 |
| | After 5 shampoo | Softness 2 | Softness 4 |
| | washes | Disentangling 2 | Disentangling 4 |

Grading:
0 = equivalent to the untreated lock
5 = very much greater than the untreated lock

The experiment shows that the sensory modifications (softness and disentangling) afforded by the composition disclosed herein were greater than those for composition 1 without boron nitride immediately after applying the compositions. Furthermore, after performing 5 shampoo washes, the remnants of the provision of softness and of the provision of disentangling afforded by the composition disclosed herein (Composition 2) was conserved and greater than that of the composition without boron nitride.

It should be noted that the levels of sheen and of disentangling were higher with the Composition 1 when it was applied to wet hair.

1. Methylheptyl cyanoacrylate monomer

| | | |
|---|---|---|
| Methylheptyl cyanoacrylate (1) | 10% | a.m. |
| Cyclopentasiloxane | 43% | |
| Cyclopentasiloxane dimethicone copolyol | 43% | |
| Boron nitride | 4% | |

(1) Sold by the company Chemence

2. Methylheptyl cyanoacrylate monomer + acetic acid

| | | |
|---|---|---|
| Methylheptyl cyanoacrylate (1) | 10% | a.m. |
| Acetic acid | 0.25% | |
| Cyclopentasiloxane | 42.75% | |
| Cyclopentasiloxane dimethicone copolyol | 43% | |
| Boron nitride | 4% | |

(1) Sold by the company Chemence

3. Ethoxyethyl cyanoacrylate monomer

| | | |
|---|---|---|
| Ethoxyethyl cyanoacrylate (1) | 10% | a.m. |
| Cyclopentasiloxane | 43% | |
| Cyclopentasiloxane dimethicone copolyol | 43% | |
| Boron nitride | 4% | |

(1) EO 460 sold by the company Tong Shen

4. Butyl cyanoacrylate monomer

| | | |
|---|---|---|
| Butyl cyanoacrylate (1) | 10% | a.m. |
| Cyclopentasiloxane | 43% | |
| Cyclopentasiloxane dimethicone copolyol | 43% | |
| Boron nitride | 4% | |

(1) B 60 sold by the company Tong Shen

5. Ethylhexyl cyanoacrylate monomer

| | | |
|---|---|---|
| Ethylhexyl cyanoacrylate (1) | 10% | a.m. |
| Cyclopentasiloxane | 43% | |
| Cyclopentasiloxane dimethicone copolyol | 43% | |
| Boron nitride | 4% | |

(1) O-60 sold by the company Tong Shen

6. Methylheptyl cyanoacrylate and ethylhexyl
cyanoacrylate monomer mixture

| | | |
|---|---|---|
| Methylheptyl cyanoacrylate (1) | 9% | a.m. |
| Ethylhexyl cyanoacrylate (2) | 1% | |
| Cyclopentasiloxane | 43% | |
| Cyclopentasiloxane dimethicone copolyol | 43% | |
| Boron nitride | 4% | |

(1) Sold by Chemence
(2) O-60 sold by the company Tong Shen

7. Methylheptyl cyanoacrylate and butyl
cyanoacrylate monomer mixture

| | | |
|---|---|---|
| Methylheptyl cyanoacrylate (1) | 7% | a.m. |
| Butyl cyanoacrylate (2) | 3% | |
| Cyclopentasiloxane | 43% | |
| Cyclopentasiloxane dimethicone copolyol | 43% | |
| Boron nitride | 4% | |

(1) Sold by Chemence
(2) B-60 sold by the company Tong Shen

What is claimed is:

1. A haircare composition comprising, in a cosmetically acceptable medium, at least one electrophilic monomer and micro- or nanoparticles of at least one solid lubricant.

2. The haircare composition according to claim 1, wherein the at least one electrophilic monomer is chosen from compounds of formula (A): 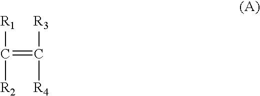
in which:
R₁ and R₂ are, independently of each other, chosen from sparingly or non-electron-withdrawing groups optionally chosen from:
hydrogen,
saturated or unsaturated, linear, branched, or cyclic hydrocarbon-based groups containing from 1 to 20 carbon atoms, optionally comprising at least one atom chosen from nitrogen, oxygen, and sulfur atoms, and optionally substituted with at least one group chosen from —OR, —COOR, —COR, —SH, —SR, —OH, and halogen atoms,
modified or unmodified polyorganosiloxane residues, and
polyoxyalkylene groups;
R₃ and R₄ are, independently of each other, chosen from electron-withdrawing groups chosen from —N(R)₃⁺, —S(R)₂⁺, —SH₂⁺, —NH₃⁺, —NO₂, —SO₂R, —C≡N, —COOH, —COOR, —COSR, —CONH₂, —CONHR, —F, —Cl, —Br, —I, —OR, —COR, —SH, —SR, —OH, linear or branched alkenyl groups, linear or branched alkynyl groups, C₁-C₄ monofluoroalkyl groups, polyfluoroalkyl groups, aryl groups, and aryloxy groups; and
R is chosen from saturated or unsaturated, linear, branched, or cyclic hydrocarbon-based groups containing from 1 to 20 carbon atoms, optionally comprising at least one atom chosen from nitrogen, oxygen, and sulfur atoms, and optionally substituted with at least one group chosen from —OR′, —COOR′, —COR′, —SH, —SR′, —OH, halogen atoms, and polymer residues, wherein R′ is chosen from C₁-C₁₀ alkyl radicals.

3. The composition according to claim 2, wherein the at least one electrophilic monomer is chosen from compounds of formula (B): 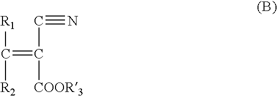
wherein:
R′₃ is chosen from hydrogen and radicals R, wherein R is chosen from saturated or unsaturated, linear, branched, or cyclic hydrocarbon-based groups containing from 1 to 20 carbon atoms, optionally comprising at least one atom chosen from nitrogen, oxygen, and sulfur atoms, and optionally substituted with at least one group chosen from —OR′, —COOR′, —COR′, —SH, —SR′, —OH, halogen atoms, and polymer residues, wherein R′ is chosen from C₁-C₁₀ alkyl radicals.

4. The composition according to claim 3, wherein the at least one electrophilic monomeris chosen from C₁-C₂₀ polyfluoroalkyl 2-cyanoacrylates, (C₁-C₁₀) alkyl cyanoacrylates, and (C₁-C₄ alkoxy)(C₁-C₁₀ alkyl) cyanoacrylates.

5. The composition according to claim 4, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

6. The composition according to claim 3, wherein the at least one electrophilic monomer is chosen from compounds of formula (F): 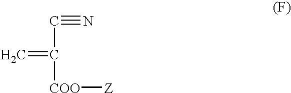
wherein Z is chosen from —(CH₂)₇—CH₃; —CH(CH₃)—(CH₂)₅—CH₃; —CH₂—CH(C₂H₅)—(CH₂)₃—CH₃; —(CH₂)₅—CH(CH₃)—CH₃; and —(CH₂)₄—CH(CH₂H₅)—CH₃.

7. The composition according to claim 1, wherein the at least one electrophilic monomer is present in the composition in an amount ranging from 0.001% to 80% by weight, relative to the total weight of the composition.

8. The composition according to claim 7, wherein the at least one electrophilic monomer is present in the composition in an amount ranging from 0.1% to 40% by weight, relative to the total weight of the composition.

9. The composition according to claim 8, wherein the at least one electrophilic monomer is present in the composition in an amount ranging from 1% to 20% by weight, relative to the total weight of the composition.

10. The composition according to claim 1, wherein the at least one electrophilic monomer is covalently bonded to at least one support.

11. The composition according to claim 10, wherein the at least one support is chosen from polymers, oligomers, and dendrimers.

12. The composition according to claim 1, wherein the cosmetically acceptable medium is anhydrous.

13. The composition according to claim 12, wherein the cosmetically acceptable medium is chosen from at least one medium chosen from organic oils, silicones, mineral oils, plant oils, waxes, C₅-C₁₀ alkanes, acetone, methyl ethyl ketone, esters of C₁-C₂₀ acids, esters of C₁-C₈ alcohols, dimethoxyethane, diethoxyethane, C₁₀-C₃₀ fatty alcohols, C₁₀-C₃₀ fatty acids, C₁₀-C₃₀ fatty amides, and C₁₀-C₃₀ fatty alkyl esters.

14. The composition according to claim 1, further comprising at least one polymerization inhibitor.

15. The composition according to claim 14, wherein the at least one polymerization inhibitor is chosen from anionic and free-radical polymerization inhibitors.

16. The composition according to claim 14, wherein the at least one polymerization inhibitor is chosen from sulfur dioxide, nitric oxide, lactone, boron trifluoride, hydroquinone and derivatives thereof, benzoquinone and derivatives thereof, catechol and derivatives thereof, anisole and derivatives thereof, pyrogallol, 2,4-dinitrophenol, 2,4,6-trihydroxybenzene, p-methoxyphenol, hydroxybutyltoluene, alkyl sulfates, alkyl sulfites, alkyl sulfones, alkyl sulfoxides, alkyl sulfides, mercaptans, and 3-sulfonene

17. The composition according to claim 16, wherein the derivatives of hydroquinone are chosen from hydroquinone monoethyl ether and tert butylhydroquinone (TBHQ).

18. The composition according to claim 16, wherein the derivative of benzoquinone is duroquinone.

19. The composition according to claim 16, wherein the derivatives of catechol are chosen from t-butylcatechol and methoxy catechol.

20. The composition according to claim 16, wherein the derivatives of anisole are chosen from methoxyanisole, hydroxyanisole, and butylhydroxyanisole.

21. The composition according to claim 14, wherein the at least one polymerization inhibitor is present in the composition in an amount ranging from 10 ppm to 20% by weight, relative to the total weight of the composition.

22. The composition according to claim 21, wherein the at least one polymerization inhibitor is present in the composition in an amount ranging from 10 ppm to 5% by weight, relative to the total weight of the composition.

23. The composition according to claim 22, wherein the at least one polymerization inhibitor is present in the composition in an amount ranging from 10 ppm to 1% by weight, relative to the total weight of the composition.

24. The composition according to claim 1, wherein the micro- or nanoparticles of at least one solid lubricant are chosen from mineral and organic particles.

25. The composition according to claim 1, wherein the micro- or nanoparticles of at least one solid lubricant are chosen from particles of:
graphite, graphite fluoride, KC₈ (potassium graphite), LiC₈ (lithium graphite),
molybdenum disulfide (MOS₂),
boron nitride,
tin disulfide (SnS₂),
BaSi₂,
silicate Si₂O₅,
titanium disulfide (TiS₂),
Li₂Si₂O₅,
cerium fluoride (CeF₃),
kaolinite,
talc,
pyrophyllite (Al₂Si₄O₁₀(OH)₂),
mica,
zirconium chloride (ZrCl₂),
muscovite,
silver sulfate (Ag₂SO₄),
montmorillonite,
silicone beads,
borax (Na₂B₄O₇),
polyterephthalates,
perfluoroalkoxy (PFA) polymers,
fluoroethylene propene (FEP) copolymer, and
nylon.

26. The composition according to claim 1, wherein the micro- or nanoparticles of at least one solid lubricant are present in the composition in an amount ranging from 0.0001% to 40% by weight, relative to the total weight of the composition.

27. The composition according to claim 26, wherein the micro- or nanoparticles of at least one solid lubricant are present in the composition in an amount ranging from 0.001% to 20% by weight, relative to the total weight of the composition.

28. The composition according to claim 27, wherein the micro- or nanoparticles of at least one solid lubricant are present in the composition in an amount ranging from 0.01% to 10% by weight, relative to the total weight of the composition.

29. The composition according to claim 1, further comprising at least one nucleophilic agent.

30. The composition according to claim 29, wherein the at least one nucleophilic agent is encapsulated.

31. The composition according to claim 1, wherein the composition is a form chosen from lotions, sprays, and mousses.

32. A process for cosmetically treating hair, comprising applying to the hair a composition comprising, in a cosmetically acceptable medium, at least one electrophilic monomer and micro- or nanoparticles of at least one solid lubricant.

33. The process according to claim 32, wherein the hair is reinforced.

34. A process for treating keratin materials, comprising applying to the keratin materials a haircare composition comprising, in a cosmetically acceptable medium, at least one electrophilic monomer and micro- or nanoparticles of at least one solid lubricant, in the presence of at least one nucleophilic agent.

35. The process according to claim 34, wherein the at least one nucleophilic agent is chosen from molecular compounds, oligomers, dendrimers, and polymers comprising at least one nucleophilic function chosen from: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, and H₂O, wherein Ph represents a phenyl group, Ar represents an aryl group, and R is chosen from C₁-C₁₀ aryl groups.

36. The process according to claim 35, wherein the at least one nucleophilic agent is water.

37. The process according to claim 34, wherein the haircare composition is applied to keratin materials that have been wetted beforehand using an aqueous solution having a pH that has been adjusted using a base, an acid, or an acid/base mixture.

38. The process according to claim 34, wherein the keratin materials are preimpregnated using at least one nucleophilic agent.

39. The process according to claim 34, further comprising reducing the keratin materials with at least one reducing agent before applying the haircare composition.

40. The process according to claim 39, wherein the at least one reducing agent is chosen from anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, and monoethanolamine thioglycolate.

41. The process according to claim 35, wherein the haircare composition further comprises at least one polymer chosen from poly(methyl methacrylate) and cyanoacrylate-based copolymers.

42. The process according to claim 35, further comprising rinsing after applying the haircare composition.

43. The process according to claim 32, comprising:
a first step comprising applying a composition comprising micro- or nanoparticles of at least one solid lubricant; and
a second step comprising applying a composition comprising at least one electrophilic monomer.

44. The process according to claim 43, wherein the first and the second step are inverted.

45. A multi-compartment kit comprising:
a first composition comprising at least one electrophilic monomer and at least one polymerization inhibitor chosen from anionic and free-radical polymerization inhibitors, and
a second composition comprising, in a cosmetically acceptable medium, micro- or nanoparticles of at least one solid lubricant.
